(19) United States
(12) Patent Application Publication
(10) Pub. No.: US 2016/0318995 A1
(43) Pub. Date: Nov. 3, 2016

(54) Randomized Humanized Antibody Libraries Engineered with Sequence and Insert Diversity

(71) Applicant: Helio Genetics Inc., East Hanover, NJ (US)

(72) Inventors: Neil Goldstein, Maplewood, NJ (US); Michael Spruyt, Mahwah, NJ (US); Frank Buquicchio, Old Tappan, NJ (US)

(21) Appl. No.: 15/144,486

(22) Filed: May 2, 2016

Related U.S. Application Data
(60) Provisional application No. 62/156,015, filed on May 1, 2015.

(51) Int. Cl. C07K 16/00; C12N 15/10; C07K 16/40

(52) U.S. Cl. CPC C07K 16/005; C07K 16/40; C12N 15/1037; C07K 2317/56; C07K 2317/565; C07K 2317/51; C07K 2317/515

(57) ABSTRACT

A phage display library is formed by a plurality of recombinant phages; each of the plurality of recombinant phages comprising an M13-derived expression vector; the M13-derived expression vector comprising a polynucleotide sequence encoding a randomized peptide, wherein the randomized peptide is a variable region of a human IgG1 heavy chain; the randomized peptide is expressed in absence of an immunoglobulin light chain protein on an outer surface of a recombinant phage of the phage display antibody library; and a protein product expressed by a recombinant phage of the phage display antibody library fuses with a human immunoglobulin constant region to form a full human IgG1 heavy chain molecule. The phage display library can be utilized to identify candidate antibodies that can bind to receptor Axl and other receptors.

The current application claims a priority to the U.S. Provisional Patent Application Ser. No. 62/56,015, filed on May 0, 2015. The current application is filed on May 2, 2016, while May 01, 2016 was on a weekend.

### FIELD OF THE INVENTION

The present invention relates to new antibody compositions, interaction systems incorporating these novel antibody compositions, and applications of using these interaction systems and the novel antibody compositions. More specifically, the present invention provides antibody libraries with great diversities as well as a process for preparing the generate the antibody libraries by way of improved antibodies binding to recombinant antigens or those expressed on cells in situ.

### BACKGROUND OF THE INVENTION

Currently monoclonal antibodies are one of the popular therapeutic drugs used for the treatment of cancer and other disease or disorders. Typically, before such antibodies can be approved for therapeutic treatments they must be modified with certain human counterparts having equivalent bioactivity, in other words they must be “humanized” in some way in order to eliminate or minimize potential immunogenicity in humans.

Currently available methods for the isolation of human antibodies with required bioactivities for therapeutic use, or for “humanizing” non-human antibodies for therapeutic use, have many limitations. For example, the grafting CDRs of the non-human antibody onto human germline frameworks selected for their structural homology to the non-human framework typically results in a substantial loss of affinity, at least some of which can be regained by making mutational adjustments in the structure based on sophisticated modeling techniques combined with an approach of trial and error. However, in many cases the original affinity of the original non-human antibody cannot be fully recovered.

In vivo methods for isolation of antigen-specific human antibodies include the screening of transgenic mice and the in vitro display methods, which include phage display, yeast and ribosome display. These methods have yielded many useful human antibodies but allow little control over epitope selection, and making the isolation of antibodies with bioactivities which are equivalent to those of non-human therapeutic lead antibodies highly uncertain. Hybridoma methods rely on the humoral immune responses of mice, and are therefore subject to the uncertainties of such responses, which may include epitope biases and failure to respond well to antigens which are homologous to host proteins. Epitope biases may arbitrarily exclude antibodies from selection which bind to certain epitopes on the native antigen surface, which do not elicit useful responses in mice. Such excluded epitopes often include epitopes which are required for desired bioactivities. In fact, selection in mice may often be biased against desired bioactivities precisely because such bioactivities may interfere with the immune response of the animal. Furthermore, mice transgenic for human immunoglobulin loci generally do not express the full complement of human diversity, and therefore the success rates for desired affinities and specificities tend to be even lower than with conventional mice. Hybridoma methods are also hampered by heavy time and labor demands. Available screening methods are highly inefficient, requiring many man-hours to screen even modest numbers of clones for desired specificities and affinities.

Display technologies offer another approach for developing human-like therapeutic antibodies and can be divided into biological display systems that employ a biological host and/or biological reactions and non-biological systems. Phage display systems offer powerful tools for the identification of antibodies that can bind and regulate the function of target proteins. Phage display is a molecular technique whereby genes are displayed in a functional form on the outer surfaces of bacteriophages by fusion to viral coat proteins. The gene product is encoded by a plasmid contained within the virus, which can be recovered and sequenced, linking the genetic information to the function of the protein. The bacterial viruses are usually members of the Ff filamentous phage family, M13, fl, fd. Foreign genes have been fused to three coat proteins, pIII, pVIII, and pVI (FIG. 1).

In general, there are three different phage display systems according to the arrangement of the coat protein genes. In the first type there is a single phage chromosome (or genome) bearing a single gene III, VIII, or VI into which is fused the foreign DNA. The resulting fusion protein is theoretically displayed on all five copies of pIII or pVI and all 2700 copies of pVIII. The phage genome encodes two copies of the coat protein in the second system, one copy fused to the foreign gene and the other wild type. The resulting phage is a mosaic since its coat contains both recombinant and wild type coat protein. The third system differs from the second in that the DNA encoding the two copies of the coat protein is on different genomes. The wild type copy is encoded by the “helper” phage while the recombinant copy is on a plasmid called a “phagemid”. Antibodies expressed on phage and binding to specific targets can be identified by in vitro selection (or biopanning; FIG. 2) while those binding to normal or diseased tissues can be found using either in vitro selection on tissue culture cells or in vivo selection. For biopanning, a display library is incubated with an immobilized target followed by extensive washing to remove non-reacting phage. Binders are usually eluted using acid or high salt and are enriched by amplification in the appropriate host cell. Biopanning usually involves 3-5 rounds in order to obtain high affinity binders. The primary structure of the antibody or antibody can then be determined by sequencing the DNA of individual clones. For the identification of in vivo binders, an initial ex-vivo selection is followed by several rounds of enrichment in the target animal. Using this approach, one can identify antibody that bind specifically to target tissues in different stages of development.

Biopanning allows the selection of target-specific antibody with affinity constants in μM to nM range. Affinity maturation of desired clones can be done through the generation of secondary libraries and subsequent binder selection under stringent conditions. In the case of antibody, secondary libraries can be designed for determining the best amino acid at each position for optimal binding, specificity and affinity. In order to achieve these types of results, secondary libraries are generated where an amino acid at any position in the sequence can change to any other amino acid. Panning of secondary libraries will identify substitutions that either do or do not affect binding. At the same time, a comparison of antibody binding to related proteins will help identify residues that confer specificity to the original target. In addition, detailed analysis of secondary binders will allow one to define an “optimal” antibody sequence which, when synthesized, can be tested for improved affinity and biological activity in the appropriate assays. When necessary, tertiary libraries can be built for further optimization. Typically, the strategy is to design tertiary libraries differing from secondary libraries in that the entire sequence is doped, so that on an average, 10-15% of the amino acids in the sequence are altered. Results of panning of tertiary libraries will usually define antibody with significant improvements in binding affinities and potency.

The principal limitations of the display technologies stem from the requirement for antigen stability in vitro, and from the lack of robust methods for affinity maturation. For selection of antibodies in vitro, antigens must be purified and immobilized on artificial surfaces, and they must remain stable in this state typically for many hours before bound antibodies can be recovered from them. Most proteins undergo varying degrees of denaturation when attached to foreign materials, and this produces strong epitope biases. Because the kinetics of binding to immobilized antigens is slow, prolonged periods of exposure are required, and this too increases the risk of denaturation. When antigens and antibodies denature, non-specific binding may occur, and if denaturation occurs after binding, binding may become irreversible and bona-fide binders may thus be lost. The problem is compounded by the fact that phage display libraries are heavily biased toward antibody chains which express well in bacteria, which may comprise only a small minority of natural human antibody chains.

The technology described in this application takes advantage of highly diverse and randomized libraries to overcome many of the limitations associated with standard display technologies and allows the generation of novel antibodies which bind to recombinant targets or those expressed in situ.

### BRIEF DESCRIPTION OF THE DRAWINGS

FIG. 1. Schematic of a typical antibody molecule expressed on Gene III of M13 phage showing orientation of an antibody within its genome. Two protein tags, FLAG and Etag are included in the vector for identification purposes.

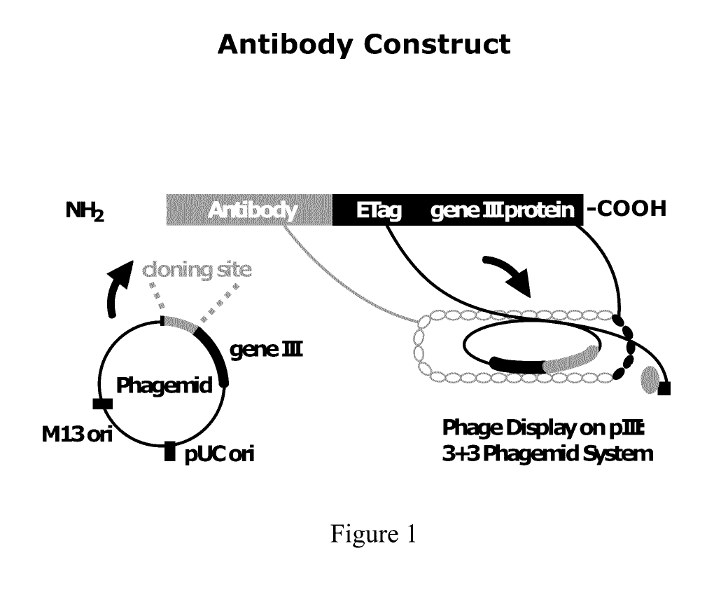

FIG. 2. Biopanning protocol. A target is first coated on an immobilized surface (e.g., microtiter plates). A phage library is added for several hr and non-binders removed by extensive washing. Target-specific phages are eluted with a low pH buffer and amplified overnight at 37oC in combination with helper phage. Usually 3-5 rounds are needed to identify target-specific binders and eliminate non-specific phage.

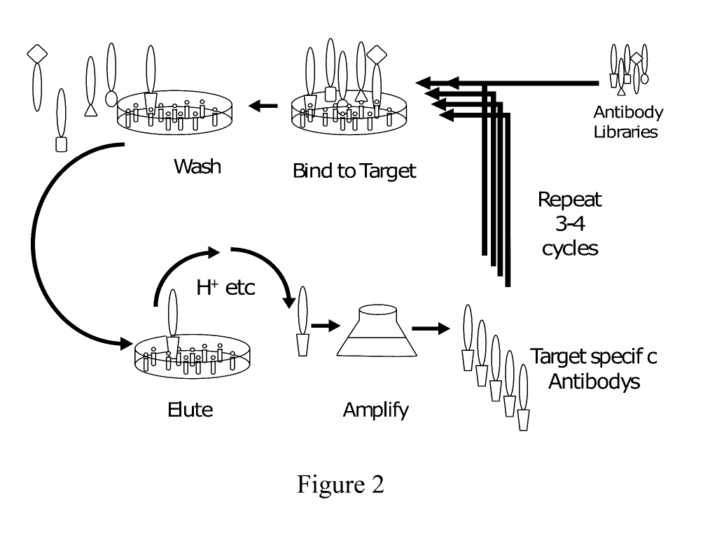

FIG. 3. Library Construction for human heavy chain randomized CDR3. A human heavy chain variable region construct is engineered with the following restriction sites flanking CDR1-3: Bsa I and Afe I for CDR1; Sac I and Xba I for CDR2; Kpn I and Sal I for CDR3. The variable region construct is inserted into the Cantab 5E plasmid and is expressed on Gene III of M13 phage. The phage library is used to pan recombinant targets or tissue culture cells.

FIG. 4. Fusion of target-specific variable region to the constant region of a human IgG1 immunoglobulin. After the phage library is panned against a target, the target-specific variable region constructs are inserted into the pFuse-CHIgG1 SS plasmid which expresses the IgG1 heavy chain constant region. Purified DNA expressing a complete target-specific, IgG1 heavy chain is subsequently used to transfect HEK-293 cells.

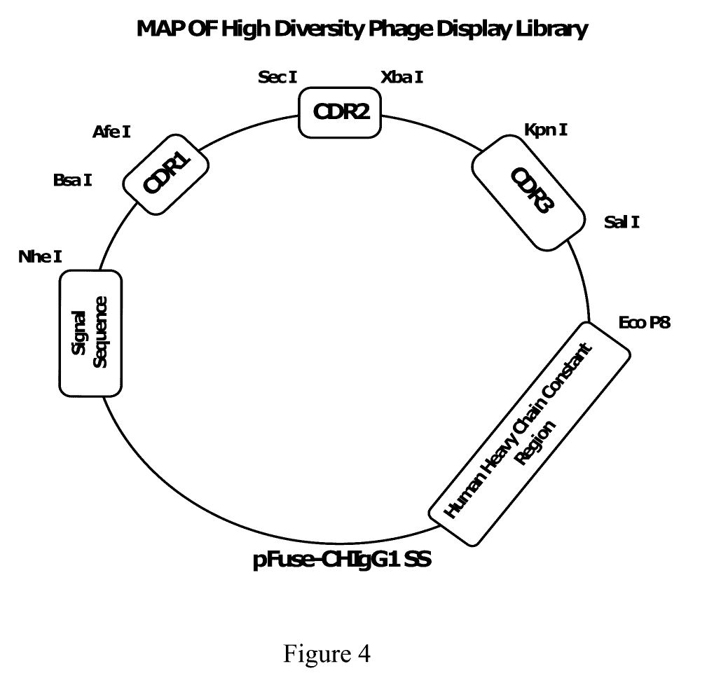

FIG. 5. Enhancement of antibody diversity through CDR3 sequence randomization and insert multiplicity. The initial antibody construct consists of a randomized CDR3 using NNK oligonucleotides and glycine/serine repeats in both CDR1 and CDR2. Target specificity derives from both the optimal amino acid sequence and the possibility of multiple inserts within CDR3.

FIG. 6. Secondary libraries for improved affinity, selectivity and potency are constructed by maintaining the target-specific sequence/insert multiplicity in CDR3 and randomizing both CDRs 1 and 2 using NNK oligonucleotides.

FIG. 7. Biological effects of anti-Axl antibody. SW480 (colorectal carcinoma), T47D (breast carcinoma), H522 (non-small cell lung cancer) and HEK-293 (human embryonic kidney) are plated in 96 well microtiter plates at 5000 cells per well in 100 ul DMEM containing 1% FBS and incubated overnight at 37C. The Axl antibody and controls are added to the appropriate wells for 72 at 37C. Cells are visualized under a phase contrast microscope and photographed (x200×).

FIG. 8. Viability effects of anti-target antibodies on SW480 cancer cells, SW480 cells (colorectal cancer) are plated in 96 well microtiter plates at 5000 cells per well in 100 ul DMEM containing 1% FBS and incubated overnight at 37C. Samples and appropriate controls (100 ul/well) are added and the plates incubated for 72 or 96 hours at 37C. Ten microliters of WST-8 (Sigma) are added to each well and the plates read at 1, 2, 3 and 4 hours in an ELISA reader at 450nM. Antibodies: Irrelevant antibody, anti-IL-8; anti-Axl antibody; cl 163, anti-MerRT; cl 36, anti-Axl; cl 8, anti-ErbB3; cl 243 and 247, anti-Axl-Gas6.

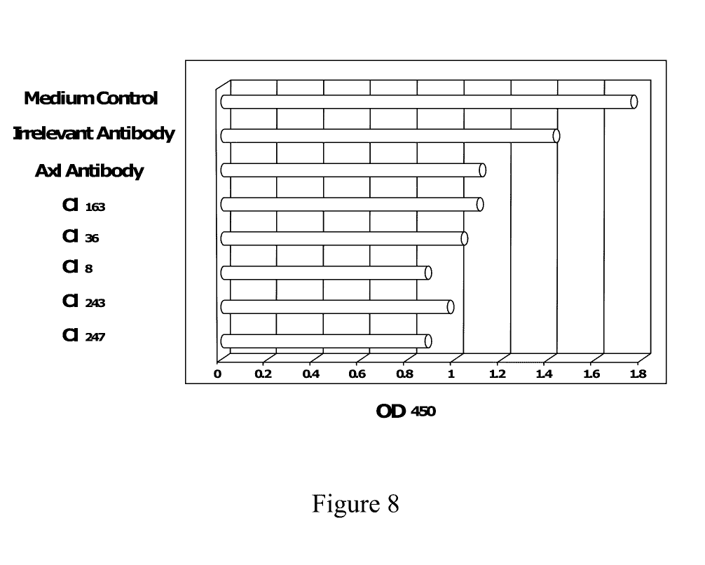

### DETAIL DESCRIPTIONS OF THE INVENTION

All illustrations of the drawings are for the purpose of describing selected embodiments of the present invention and are not intended to limit the scope of the present invention.

This invention provides for a novel means to generate improved antibodies binding to recombinant antigens or those expressed on cells in situ. The general approach is designated “RAE” for Randomized Antibody Engineering. The invention relates to new antibody compositions, interaction systems incorporating these novel antibody compositions, and methods of using these interaction systems and the novel antibody compositions. Whereas the majority of sequences in the pre-screened library appear to consist of a single insert in the CDR3 region, many of the target-specific binders have multiple inserts in the same region suggesting that the interaction between the library and target selects for the “best” binders including those longer sequences which may be relatively rare in the library.

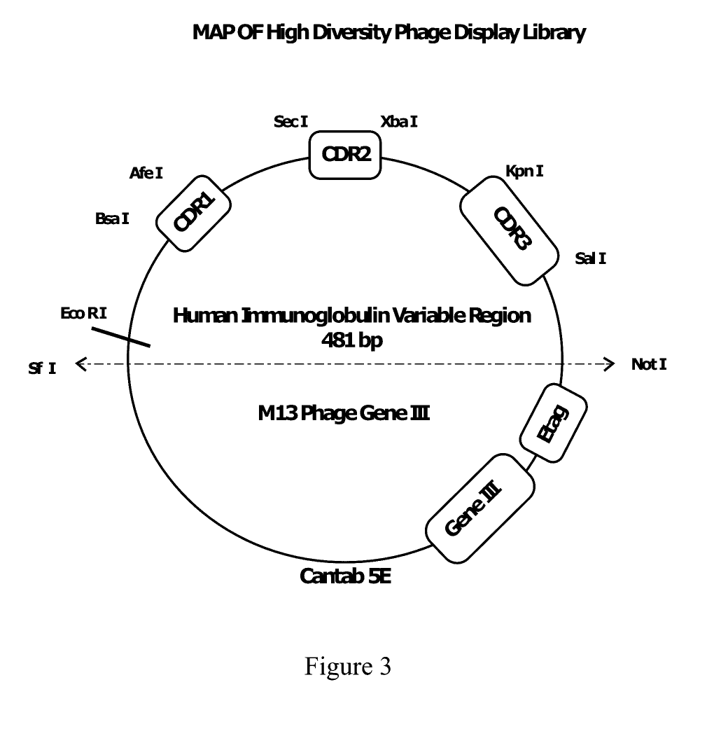

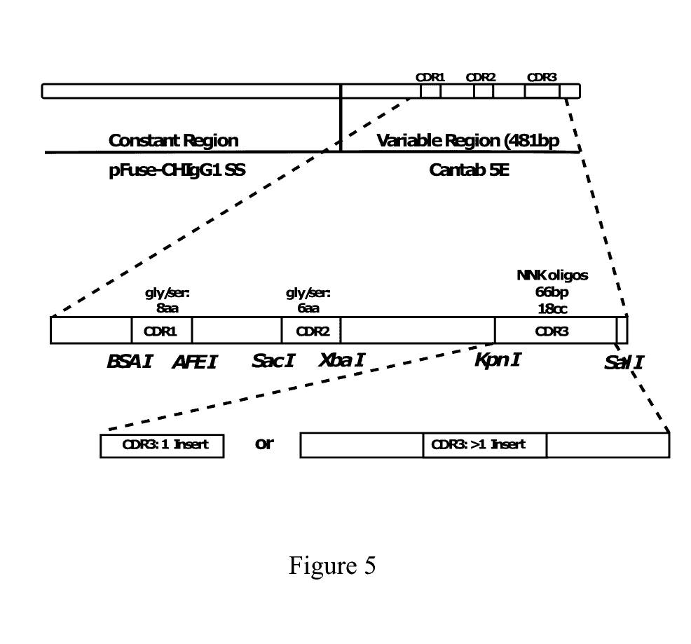

In first aspect of the present invention antibody libraries are generated by phage display wherein the CDR3 of a human IgG1 heavy chain variable region is totally randomized using NNK oligonucleotides with a Kpn I restriction site at the 5′ end and a Sal I site at the 3′ end (FIG. 3, 4). Unexpectedly, these two restriction sites also allow the generation of randomized CDR3 regions containing multiple inserts (anywhere from 1-7 inserts within the CDR3; FIG. 5). Therefore, diversity derives from both sequence and insert randomization. The initial size of the CDR3 insert is 66 oligonucleotides (including the restriction sites coding for 18 amino acids) and the entire variable region insert (including CDR1, 2 and 3 plus framework) which is fused to M13 Gene III is 481 oligonucleotides. The amino acid sequence of CDR1 and 2 is initially comprised of glycines and serines. After panning, target-specific binders are identified and fused to a human immunoglobulin constant region to form a complete target-specific IgG1 heavy chain molecule. Target binding sequences against several antigens and antigens on human tissue culture cells have been shown to be novel and not related to the normal human repertoire.

A second aspect of this invention allows the generation of secondary libraries with improved affinity, selectivity and potency by identifying the optimal amino acids for target binding within the CDR3. To determine optimal amino acid requirements within the CDR3 region of the antibody, a secondary library can be generated from “doped” oligonucleotides so that half of the amino acid residues (on average) per antibody.

Panning of this library will identify substitutions that either affect or have no effect on binding to the target. Amino acids optimal for binding are selected during panning. This includes residues at randomized positions where any amino acid is allowed by the design of the library.

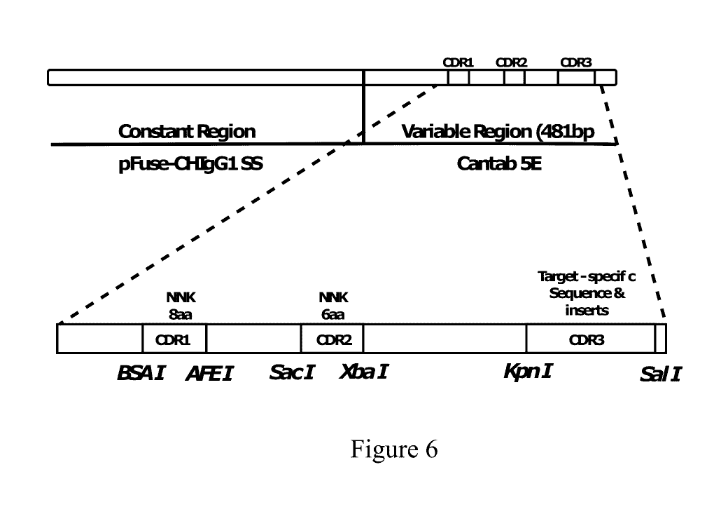

A third aspect of this invention allows the generation of secondary libraries with improved affinity, selectivity and potency by maintaining the amino acid composition of the CDR3 and randomizing either or both the CDR1 or 2 of the same heavy chain molecule using methods described above. All target specific variable regions are later fused to a human immunoglobulin constant region to form a complete target-specific IgG1 heavy chain molecule (FIG. 6)

A fourth aspect of this invention allows the generation of light chain libraries with randomized CDR1, 2 and/or 3 for identification of specific target binders. All light chain variable region binders are further fused to a human immunoglobulin constant region to form a complete target-specific lambda light chain

A fifth aspect of this invention allows the generation of whole, fully functional IgGl, lambda, target specific antibodies by co-transfect ng optimized heavy and light chain binders into expression systems such as CHO, HEK-293 and NS 1-0.

In the present invention, antibody diversity within the phage display library is generated by complete randomization of the amino acid sequences of CDRs 1-3 on both the heavy and light chain variable regions. In addition, diversity is further generated by the number of inserts within each CDR which is a consequence of the target being panned by the library. In addition, there exists the possibility of generating CDRs with multiple inserts which add to the diversity of the libraries.

### DESCRIPTION OF EMBODIMENTS

In the present invention, “antibody” refers to a protein functionally defined as a binding protein and structurally defined as comprising an amino acid sequence that is recognized by one of skill as being derived from the framework region of an immunoglobulin encoding gene of an animal producing antibodies. An antibody can consist of one or more antibodies substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer, is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. For example, pepsin digests an antibody below the disulfide linkages in the hinge region to produce F(ab)′2, a dimer of Fab which itself is a light chain joined to VH-CH1 by a disulfide bond. The F(ab)'2 may be reduced under mild conditions to break the disulfide linkage in the hinge region thereby converting the (Fab′)2 dimer into an Fab′ monomer. The Fab′ monomer is essentially an Fab with part of the hinge region. For the purposes of this patent, an antibody is also defined as a single heavy or light chain comprising a framework region and 3 CDRs.

“Antigen” refers to substances which are capable of inducing a specific immune response and of reacting with the products of that response, including antibodies. Antigens may be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” may be used to refer to any substance to which an antibody binds, or for which antibodies are desired, regardless of whether the substance is immunogenic. For such antigens, antibodies may be identified by recombinant methods, independently of any immune response.

“Antibody library” refers to a library of oligonucleotides which is randomized in one or more CDRs on a human IgG1 heavy chain or a human lambda light chain and expressed on Gene III of M13 phage.

“Binding” refers to the adherence of an antibody to an antigen one another, for example, enzymes to substrates, antibodies to antigens, DNA strands to their complementary strands. Binding occurs because the shape and chemical natures of parts of the molecules surfaces are complementary.

“Complementarity-determining region” and “CDR” refer to the art-recognized term as exemplified by the Kabat and Chothia CDR definitions also generally known as hypervariable regions or hypervariable loops (Chothia and Lesk (1987) J. Mol. Biol. 196: 901; Chothia et al. (1989) Nature 342: 877; E. A. Kabat et al., Sequences of Proteins of Immunological Interest (National Institutes of Health, Bethesda, Md.) (1987)). Variable region domains typically comprise the amino-terminal approximately 105-115 amino acids of a naturally-occurring immunoglobulin chain (e.g., amino acids 1-110), although variable domains somewhat shorter or longer are also suitable for forming single-chain antibodies.

“Epitope” refers to that portion of an antigen or other macromolecule capable of forming a binding interaction that interacts with the variable region binding pocket of an antibody. Typically, such binding interaction is manifested as an intermolecular contact with one or more amino acid residues of a CDR.

“Expression vector” includes vectors which are capable of expressing nucleic acid sequences contained therein, i.e., any nucleic acid sequence which is capable of effecting expression of a specified nucleic acid code disposed therein (the coding sequences are operably linked to other sequences capable of effecting their expression). Some expression vectors are replicable in the host organism either as episomes or as an integral part of the chromosomal DNA. A useful, but not a necessary, element of an effective expression vector is a marker encoding sequence—i.e. a sequence encoding a protein which results in a phenotypic property (e.g. tetracycline resistance) of the cells containing the protein which permits those cells to be readily identified. Expression vectors are frequently in the form of plasmids or viruses. However, the invention is intended to include such other forms of expression vectors which serve equivalent functions and which may, from time to time become known in the art.

“Insert” refers to the number of repetitive sequences within CDRs of the heavy and/or light chain of the antibody molecule. For example, the heavy chain CDR3 is bounded by a Kpn I site on the 5′ end and a Sal I site on the 3′ end.

“Host cell” refers to a prokaryotic or eukaryotic cell into which the vectors of the invention may be introduced, expressed and/or propagated. Typical prokaryotic host cells include various strains of E. coli. Typical eukaryotic host cells are yeast or filamentous fungi, or mammalian cells, such as Chinese hamster ovary cells, murine NIH 3t3 fibroblasts, or HEK-293 cells.

“Library” means a collection of nucleotides sequences, e.g., DNA, encoding antibodies within clones; or a genetically diverse collection of antibody antibodies.

“Phenotype” refers to a physical (e.g., pigment, or cell shape) and/or metabolic property of a cell which can be measured or exploited in some fashion and which is affected by the reporter gene.

“RAE” or Randomized Antibody Engineering refers to the technique used to generate randomized antibody libraries with high diversity based on sequence and insert randomization.

“Target” may be used to refer to the molecule to which an antibody binds; “target” may herein be used synonymously with “antigen”.

“Vector” refers to a plasmid or phage or virus or vector, for expressing a polypeptide from a DNA (RNA) sequence. The vector can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate translation initiation and termination sequences. Structural units intended for use in yeast or eukaryotic expression systems may include a leader sequence enabling extracellular secretion of translated protein by a host cell.

Each of the above terms is meant to encompass all that is described within the present application unless the context dictates otherwise.

The systems and methods of the present invention overcome many limitations of current methods for the isolation of human antibodies which bind to antigens of choice with high affinities and specificities. Human antibodies are required for many therapeutic applications. However, current methods for obtaining human antibodies with required bioactivities for therapeutic use are often unreliable. These methods include isolating antigen-specific hybridomas from human antibody-producing transgenic mice, and isolating antigen-specific human antibody genes from libraries displayed on bacteriophage, cells, or ribosomes by biopanning.

Hybridoma technologies rely on the humoral immune responses of mice, and are therefore subject to the uncertainties of such responses, which may include epitope biases and failure to respond well to antigens which are homologous to host proteins. Epitope biases may arbitrarily exclude antibodies from selection which bind to certain epitopes on the native antigen surface, which do not elicit useful responses in mice. Such excluded epitopes often include epitopes which are required for desired bioactivities. In fact, selection in mice may often be biased against desired bioactivities precisely because such bioactivities may interfere with the immune response of the animal. Hybridoma technologies are also hampered by time and labor demands. Available screening methods are highly inefficient, requiring many man-hours to screen even modest numbers of clones for desired specificities and affinities, compared to the numbers of clones that can be screened by in vitro methods. Also, mice transgenic for human immunoglobulin loci generally do not express the full complement of human diversity, and therefore the success rates for desired affinities and specificities tend to be lower than with conventional mice.

The systems and methods of the present invention takes advantage of complete randomization of CDRs 1-3 on the heavy and light chain of a reference antibody to identify target specific binders with biological activity. The crux of the invention is to allow “biology” to make a decision about what CDR sequence(s) is important for binding. Binding is determined by panning of a recombinant target or target-expressing tissue culture cells with the library. A high level of diversity is obtained from both the random nature of the amino acid sequences of the heavy and light chain CDRs plus the potential for multiple inserts within the CDRs themselves.

Antibodies are antibodies comprising at least a heavy chain variable region and a light chain variable region that together specifically bind and recognize an antigen, the variable regions being specified by immunoglobulin genes. Recognized immunoglobulin genes include the kappa, lambda, alpha, gamma, delta, epsilon, and mu constant region genes, as well as the immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition.

Structurally, the simplest antibody (IgG) comprises four polypeptide chains, two heavy (H) chains and two light (L) chains inter-connected by disulphide bonds . Each chain has a constant region (C) and a variable region (V). Each chain is organized into a series of domains. The light chains have two domains, corresponding to the C region and the other to the V region. The heavy chains have four domains, one corresponding to the V region and three domains (1,2 and 3) in the C region. The antibody has two arms (each arm being a Fab region), each of which has a VL and a VH region associated with each other. It is this pair of V regions (VL and VH) that differ from one antibody to another (owing to amino acid sequence variations), and which together are responsible for recognizing the antigen and providing an antigen binding site. In even more detail, each V region is made up from three complementarity determining regions (CDR) separated by four framework regions (FR). The CDRs are the most variable part of the variable regions, and they perform the critical antigen binding function. The CDR regions are derived from many potential germ line sequences via a complex process involving recombination, mutation and selection.

Antibody diversity refers primarily to the diversity of antibody binding sites in a group or library of antibody-encoding genes. The specificity of an antibody's binding site is determined by its CDRs (complementarity determining regions). Light chains and/or heavy chains of an antibody have 3 CDRs (CDR1, CDR2, and CDR3). An immunoglobulin light chain or heavy chain variable region consists of a “framework” region interrupted by the three CDRs (hypervariable regions). The extent of the framework region and CDRs have been precisely. The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs. The CDRs are primarily responsible for binding to an epitope of an antigen.

The diversity of binding sites for a group or library of antibodies is thus a reflection of the diversity of sequence in the CDRs, the degree of combinatorial combinations of these CDRs in the light and heavy chains, and the degree of combinatorial combinations of the light and/or heavy chains in a group or library of antibodies. All three of these variables may be manipulated to generate a group or library of antibodies having a desired level of diversity. For example, the diversity of an antibody library can be increased by shuffling the heavy and light chain genes or by altering the CDRs of the cloned antibody genes. Mutagenesis of the CDRs can be done by error-prone replication, replicative incorporation of degenerate oligonucleotides, or by the methods of chemical or UV mutagenesis. PCR may also be used to create amino acid sequence variants of the polynucleotides of the invention. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide or protein at the position specified by the primer. The product DNA fragments replace the corresponding region in the plasmid and this gives the desired amino acid variant. In another embodiment, error prone PCR may be used to generate amino acid variants in the polynucleotides that encode the antibodies.

There are many expression systems for producing the antibodies of the invention that are well known to those of ordinary skill in the art. Typically, the polynucleotide that encodes the polypeptide is placed under the control of a promoter that is functional in the desired host cell. An extremely wide variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites, enhancers, operators, and the like are also optionally included.

Constructs that include one or more of these control sequences are termed “expression cassettes.” Accordingly, the nucleic acids that encode the joined antibodies are incorporated for the desired level of expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems. The particular promoter system is not critical to the invention, any available promoter that functions in prokaryotes can be used. Epitope tags can also be added to recombinant proteins to provide convenient methods of isolation, e.g., c-myc, HA-tag, 6-His tag, maltose binding protein, VSV-G tag, anti-DYKDDDDK tag, or any such tag.

Similarly, for expression of the antibodies of the invention in eukaryotic cells, transcription and translation sequences that function in the particular eukaryotic species are required. For example, eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Either constitutive or regulated promoters can be used in the present invention. Regulated promoters can be advantageous because the concentration of heterologous protein in the host cell can be controlled. An inducible promoter is a promoter that directs expression of a gene where the level of expression is alterable by environmental or developmental factors such as, for example, temperature, pH, anaerobic or aerobic conditions, light, transcription factors and chemicals.

The antibodies of the invention can be expressed intracellularly, or can be secreted from the cell. Intracellular expression often results in high yields. If necessary, the amount of soluble, active polypeptide may be increased by performing refolding procedures. Antibodies of the invention can be expressed in a variety of host cells, including E. coli, other bacterial hosts, yeast, and various higher eukaryotic cells such as the COS, CHO and HeLa cells lines and myeloma cell lines. The host cells can be mammalian cells, insect cells, or microorganisms, such as, for example, yeast cells, bacterial cells, or fungal cells. Once expressed, the recombinant antibodies can be purified according to standard procedures of the art, including ammonium sulfate precipitation, affinity columns, column chromatography, gel electrophoresis and the Substantially pure compositions of at least about 90 to 95% homogeneity are preferred, and 98 to 99% or more homogeneity are most preferred.

To facilitate purification of the antibodies of the invention, the nucleic acids that encode the antibodies can also include a coding sequence for an epitope or “tag” for which an affinity binding reagent is available. Examples of suitable epitopes include the myc and V-responder genes; expression vectors useful for recombinant production of fusion antibodies having these epitopes are commercially available vectors pcDNA3.1/Myc-His and pcDNA3.1/V5-His are suitable for expression in mammalian cells).

In one embodiment, the novel antibody compositions of the invention comprise antibodies in complexes with other components of an interaction assay. In this manner, novel antibodies with the appropriate binding specificities are determined. Further, the novel antibodies are comprised of human IgG1 heavy or lambda light chains with randomized CDRs which have been optimized for antigen binding. In this manner, these antibodies can be used for diagnostic or therapeutic purposes when the antigen is involved in a specific disease indication.

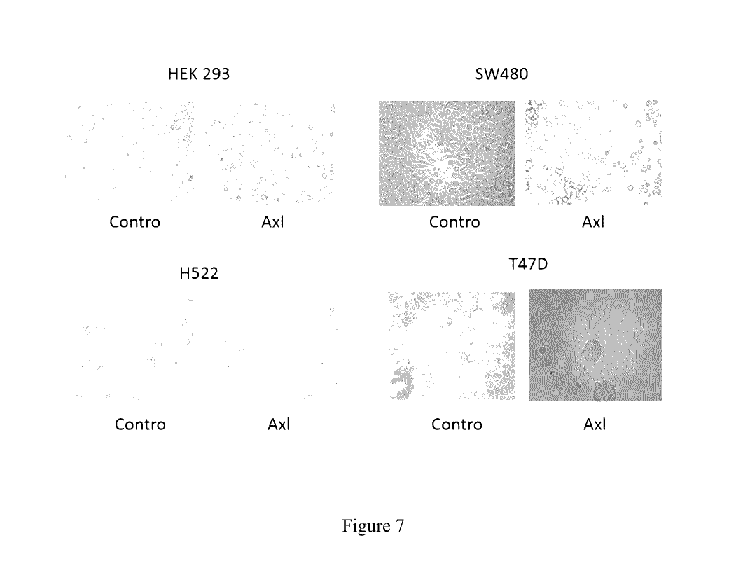

In another embodiment of the present invention, it involves the identification and characterization of human IgG1 heavy chains with binding specificity for the tyrosine kinase receptor Axl which has utility as a diagnostic or therapeutic (FIG. 7, 8).

### EXAMPLE 1

### Generation of a Randomized Antibody Engineered (RAE) Phage Display Library and Phage Rescue

DNA fragments coding for 18 random amino acids of the human heavy chain CDR3 were generated in the following manner. A 78-base oligonucleotide was synthesized to contain the sequence (NNK)18, where N=A, C, T, or G, and K=G or T (Oligo 1).

Oligo 1:
TACTGTGGTACCNNKNNKNNKNNKNNKNNKNNKNNKNNKNNKNNKNNKNN
KNNKNNKNNKNNKNNKGTCGACCAGGGA).

The oligo contains sufficient nucliotides at the 5′ and 3′ to encode the restriction sequences Kpn I and Sal I at the 5′ and 3′ end respectively. This oligonucleotide was used as the template in PCR amplification along with two shorter 20mer oligonucleotide primers containing the appropriate restriction sites and random bases at the 5′ ends to permit efficient cutting with restriction enzymes, both of which were biotinylated at their 5′ ends. The resulting products were purified and concentrated with QIAquick spin columns (Qiagen), then digested with Kpn I and Sal I in a double digest. Streptavidin-agarose (GIBCO) was added to the digestion mixture to remove the cleaved ends of the PCR product as well as any uncut DNA. The resulting was again purified over QIAquick spin columns. The phagemid pCANTAB5E (Pharmacia) containing the anti-HIV gp120 antibody heavy chain variable region (GenBank: AAB28805.1) modified to contain Kpn I and Sal I sites at the 5′ and 3′ end of the CDR3, was digested with Kpn I and Sal I followed by Shrimp Alkaline phosphatase (NEB) treatment. The vector and insert were ligated overnight at 15 □C. The ligation product was purified using QIAquick spin columns (Qiagen) and electroporations were performed at 1500 V in an electroporation cuvette (0.1 mm gap; 0.5 mL volume) containing 12.5 μg of DNA and 500 μL of Escherichia coli strain TG1 electrocompetent cells ((F′ traD36 lacIq (lacZ)M15 proAB) / supE (hsdM-mcrB)5 rk-mk-McrB-) thi (lac-proAB)). Immediately after the pulse, 12.5 mL of pre-warmed (40 C) 2xYT-G medium containing 2% glucose (2xYT-G) was added and the transformants were grown at 37 C for 1 h. Cell transformants were pooled, the volume measured, and an aliquot was plated onto 2xYT-G containing 100 μg/mL ampicillin (2xYT-AG) plates to determine the total number of transformants.

Phage rescue was carried out using the standard phage preparation protocol with the following changes. Five individual recombinant cell libraries, with a total diversity of 1.6×1010, were combined and grown to OD600=0.5 in 2xYT-AG at 30 C with shaking (250 rpm). M13K07 helper phage was then added [Multiplicity of Infection (MOI)=15], and the cells were incubated for 30 min at 37 C without shaking, followed by 30 min at 37 C with shaking (250 rpm). Following infection, cells were pelleted and the supernatant containing the helper phage was discarded. The cell pellet was resuspended in the initial culture volume of 2xYT without glucose and containing 100 mg/mL ampicillin and 50 mg/mL kanamycin (2xYT-AK) and grown overnight at 30° C. with shaking (250 rpm). The cells from the overnight culture were pelleted at 3000×g for 30 min at 4° C. and the supernatant containing the phage was recovered. The phage were precipitated by adjusting the solution to 4% PEG, 500 mM NaCl and chilled on ice for 1 h. The precipitated phage were pelleted by centrifugation at 10,000×g for 30 min. The precipitated phage pellet was resuspended in phosphate-buffered saline ( 1/100 of the initial culture volume) and passed through a 0.45 p.m filter. The phage were titered by infecting TG1 cells. The phage titer for the 40-mer peptide library was 4×1013 cfu/mL. The phage titer for the 20-mer library was XX.

To amplify the library, the transformants were inoculated into four liters of 2xYT-AG medium and allowed to grow until the OD600 increased approximately 400 times. The cells were pelleted by centrifugation at 3000×g for 20 min, then resuspended in 40 mL 2xYT-AG to which glycerol was added to a final concentration of 8%. The library was stored at −80C.

### EXAMPLE 2

### Generation of an Antibody Binding to the Receptor Tyrosine Kinase Axl

AXL is a member of the TAM (Tyro3, AXL, MER) receptor tyrosine kinase (RTK) family and was originally isolated as a transforming gene in cells from patients with chronic myeloproliferative disorders. This subfamily is characterized by an extracellular domain, consisting of two immunoglobulin-like domains and two fibronectin type III (FNIII) motifs, a single transmembrane domain and an intracellular tyrosine kinase domain . AXL and MER share the vitamin K-dependent ligand GAS6 (growth-arrest-specific 6). Binding of GAS6 to AXL results in receptor dimerization, autophosphorylation of the tyrosine residues 779, 821 and 866 and recruitment of adaptor molecules. In other cases, ligand-independent dimerization and activation can also occur. AXL is ubiquitously expressed and detected in a wide variety of cells such as macrophages, platelets and endothelial cells. Subsequent to its identification in chronic myelogenous leukemia, overexpression of AXL has been reported in a wide variety of cancers, such as breast, lung and brain tumors.

To identify antibodies binding to Axl, individual wells of a 96-well microtiter plates are coated with 100-200ng of recombinant Axl (Acro Biosystems) in 100 ul of coating buffer [50mM sodium bicarbonate, pH 8.5). Plates are incubated overnight at 4 C. The next day, unbound antigen is removed and the coated wells are blocked with 300 μl of NFM-PBS (PBS containing 4% non-fat milk) for one hr at room temperature. The plates are then washed 3 times with PBS. The phage libraries are thawed and mixed with 0.1 volume of NFM-PBS, 100 ul of each library is added to the antigen-coated wells and the plates are incubated for 1-3 hr at room temperature. Each well is washed 13 times with NFM-PBS and the phage eluted with 100 μl of glycine-HCL (50mM glycine buffer with 0.1% BSA, pH 2.2) following a five min incubation. The eluted phage from each library are pooled, neutralized with 100 μl of 1M Tris-HCl (pH 8.0) and added to 10 ml of log phase E coli TGla and amplified in 2x YT-2% glucose medium for one hr at 37C. Helper phage (M13K07; >5×1010 phage/ml) and ampicillin (50 ug/ml) are then added and the cells are incubated for an additional hr at 37 □C. The cells are pelleted at 1,000×g 10 min, resuspended in 2x YT medium containing ampicillin (50 ug/ml) and kanamycin (50 ug/ml) and incubated overnight at 37C. The next day, the infected bacterial cells are centrifuged at 2,500×g 10 min and the pellet discarded. The supernatant contains the phage and is precipitated with ¼ volume of PEG-8000 (30% PEG-8000 in 1.6 M NaCl) by incubating on ice for 1 hr. The precipitant is centrifuged at 10,000 RPM (g-force needed)10, 000×g 30 min at 4C for 30 min and the phage pellet resupended in about 1 ml of NFM-PBS. The phage is then used for the next round of panning. Normally, 3-4 rounds of panning are done for secondary libraries. Usually, 96 random clones are picked from rounds 3 and 4 and grown in 96-well cluster plates as a master stock and used for sequencing.

### EXAMPLE 3

### Biopanning of Human Cancer Cells

Cells are grown in appropriate growth medium or under special defined conditions in 6 well plates to near confluency. Cells can be panned in situ or fixed with 3.7% neutral buffered formalin for 15′ at room temperature. For viable cell panning, plates are blocked with 4% non-fat milk in Dulbecco's PBS for 60 minutes at 37C. 250 ul of the antibody library is mixed with 250 ul of Dulbecco's PBS and then added to the cells for 90 minutes at room temperature. The library is removed and the plates washed 5× with DPBS. 500 ul of glycine-HCl is added and neutralized with 500 ul of Tris, pH 8.0. The eluted phage is processed as described above for recombinant proteins.

For formalin-fixed cells, the plates are washed 3× with Dulbecco's PBS and plates blocked with NFM-PBS for 60 minutes at room temperature. 250 ul of the antibody library is mixed with 250 ul of NFM-PBS and added to each well for 2hr at room temperature and processed as for the viable cell pan.

### EXAMPLE 4

### Assay for Antibody Binders.

A master stock is prepared by isolating individual colonies and growing them overnight at 37C in 500 μl of 2xYT-AG. Forty μl of the master stock is transferred from each master to another set of cluster tubes containing 500 ul of 2x YT-AG and helper phage. The tubes are incubated at 37C with constant shaking for two hr. The cultures are centrifuged at 2500×g at 4C for 20 min, the supernatant is discarded, the bacterial pellet resuspended in 400 μl of 2x YT-AK and the plate incubated overnight at 37C. At that time, the cells are removed by centrifugation at 2500×g and the supernatants transferred to a new set of cluster tubes and used in an ELISA.

For antibodies against a recombinant protein, each well of a 96 well microtiter plate is coated with 100 ul of target overnight at 4C. The wells are blocked with PBS containing 4% non-fat milk for 1 hr at room temperature. Phage is added at 100 μl/well and the plates incubated for 3 hr at room temperature. After washing 3× with PBS-0.5%

Tween 20, plates are probed with an anti-M13 antibody conjugated to horseradish peroxidase (1:3000 in PBS-NFM) for 1 hr at room temperature followed by addition of 50 μl of TMB for 15-30 min at room temperature. The reaction is stopped by the addition of 50 ul 1N HCl. The OD is measured at 450 nM.

For antibodies to targets expressed in situ, cells are grown to near confluency and either fixed with 10% neutral buffered formalin or used without fixation. For fixed cells, plates are washed 3× with distilled water and blocked with PBS-NFM for 60′. For viable cells, plates are blocked with 4% non-fat milk in an isotonic buffer such as Dulbecco's PBS or Hanks Balanced SalIt Solution. Following the blocking step, the phage preparations are tested as above for recombinant proteins.

### EXAMPLE 5

### Preparation of DNA for Transfections

DNA for transient transfections is done using a Qiagen plasmid kit which uses anion-exchange tips for efficient purification of plasmid DNA. Up to 10 mg high-copy plasmid DNA can be purified from culture. Briefly, plasmids are grown in bacteria (e.g., HB101) and the cells lysed with an alkaline buffer. The DNA is then purified over a silica based column and diluted in sterile water. The DNA is quantitated in a spectrophotometer using the ratio 280/260nm and used for transient transfection

### EXAMPLE 6

### Transient Transfection Protocol in HEK-293 Cells

Transient transfections are done in HEK-293 cells. Briefly, 293 cells are grown overnight at 37C in 24 well microtiter plates containing 0.5 ml DMEM supplemented with 7.5% fetal bovine serum (DMEM-7.5). DNA is transfected into 293 cells using

Lipofectamine 3000 (Life Technologies) as per the manufacturer's directions. Briefly, 5 microliters of DNA are added to 30 ul serum-free OptiMem medium containing lul Lipofectamine reagent and mixed with 30 ul OptiMem containing 2ul Lipofectamine 3000. The mixture is incubated for 10 minutes at room temperature and then added to the 293 cells. After 18 hours, lml of DMEM-7.5 is added to each well and the plates incubated for an additional 3 days at 37C when it is tested for Ig production.

### EXAMPLE 7

### ELISA for Ig Production

96 well microtiter plates are coated with 200 ng/ml goat anti-human IgG, lambda (Invitrogen) overnight at 4C. Plates are then blocked with PBS containing 4% non-fat milk for 60′ at room temperature. 100 ll of supernatant from each transfected well is added in triplicate to the plate and incubated for 2 hours at room temperature. Plates are washed three times with PBS-Tween 20 (0.5%). 100 ll of goat anti-human IgG conjugated to horse radish peroxidase (Sigma) is added to each well and the plates incubated for 60 minutes at room temperature. After washing 5× with PBS-Tween, 50 ul of TMB (Sigma) is added to each well and incubated for 15-30 minutes at room temperature. The reaction is stopped with 50 ll HCl and the plates read in an ELISA reader (BioTek) at 450nM.

### EXAMPLE 8

### Viability Assay

Tissue culture cells are plated in 96 well microtiter plates at 5000 cells per well in 100 ll DMEM containing 1% FBS and incubated overnight at 37C. Multiples of the Ig containing samples and appropriate controls (100 l1/well) are added and the plates incubated for 72 or 96 hours at 37C. Ten microliters of WST-8 (Sigma) are added to each well and the plates read at 1, 2, 3 and 4 hours in an ELISA reader at 450nM. WST-8 allows very convenient assays by utilizing Dojindo's highly water-soluble tetrazolium SalIt. WST-8 [2-(2-methoxy-4-nitrophenyl)-3-(4-nitrophenyl)-5-(2,4-disulfophenyl)-2H-tetrazolium, monosodium SalInt] produces a water-soluble formazan dye upon reduction in the presence of an electron mediator. WST-8 is reduced by dehydrogenases in cells to give an orange colored product (formazan), which is soluble in the tissue culture medium. The amount of the formazan dye generated by dehydrogenases in cells is directly proportional to the number of living cells.

### EXAMPLE 9

### High Expression Transfection

The Expi293 cell line (Life Technologies) is used to obtain milligram quantities of each antibody for further biological characterization. These cells are variants of the 293 cell line that has been adapted to high-density suspension growth in Expi293TM Expression

Medium. The 293 cell line is a permanent line established from primary embryonic human kidney transformed with sheared human adenovirus type 5 DNA (1,2). The E1A adenovirus gene expressed in 293 cells participates in transactivation of some viral promoters, allowing these cells to produce very high levels of protein in suspension culture. Transfection of Expi293 cells are done as per the manufacturer's instructions. Briefly 7.5×107 viable cells are seeded in 125 ml sterile flasks containing 25.5 ml of Expi293 expression medium. In tube 1, 30 ll of purified DNA is added to 1.5 ml of OptiMem medium and in tube 2, 80 ll of ExpiFectamine 293 reagent is added to 1.5 ml OptiMem. After a 5 minute incubation, the contents of tubes 1 and 2 are mixed and incubated for 20-30 minutes at room temperature. Add the DNA complexes (3 ml) to each flask and incubated overnight at 37C on a shaker (125 rpm). After 18 hours, add 0.15 ml of Enhancer 1 and 1.5 ml of Enhancer 2 to each well. Ig production is tested after 7 days by ELISA. The amount of Ig expression is quantitated by a titration ELISA against a purified IgG1 control. Antibody containing supernatant is clarified by spinning at 5000×g for 10′ for use in biological assays.

### EXAMPLE 10

### Generation of Secondary doped CDR3 Libraries

The desired number of amino acids mutations was introduced in the target-specific CDR3 at the codon level when the synthetic DNA template was produced. For example if 45% amino acid change is desired (i.e., 9 changes /20 amino acids), then in general 60% change at the codon level is needed due to the redundancy of the genetic code (efficiency factor of 0 .75). Per position this translates to 20% doping at the level of DNA synthesis. At the DNA synthesis level the 20% doping is requested by the following designation:

| | | | | |
|---|---|---|---|---|
| A | 80% A, | 6.6% C | 6.6% G, | 6.6% T |
| C | 6.6% A | 80% C | 6.6% G | 6.6% T |
| G | 6.6% A | 6.6% C | 80% G | 6.6% T |
| T | 6.6% A | 6.6% C | 6.6% G | 80% T |

The A, C, T, G (underlined and in bold) are the original bases in the parental sequences. When the clones from cell libraries are sequenced and the number of amino acid mutations are examined per CDR, the average number of changes correlates to the desired value. After the synthetic template was obtained, the DNA was ligated to the pCANTBA5E phagmid vector to produce the cell library in the TGI strain as previously described. Phage rescue was carried out to produce the phage library used in the panning experiments.

### EXAMPLE 11

### Generation of Secondary Libraries with Fixed CDR3 Sequence and Random CDR1 and/or CDR2 Sequences

Randomized CDR1/2 libraries with target-specific CDR3 were generated using NNK oligonucleotides appropriate for the size of the CDRs (CDR1=8amino acids; CDR2=6 amino acids) as described in Example 1 except that the restriction sites were as follows: For CDR1, the 5′ restriction site is Bsa I and the 3′ site is Afe I; for CDR2, the 5′ site is Sac I and the 3′ site is Xba 1. Library production, electroporation, amplification and phage rescue is performed in the same manner as described in Example 1.

### EXAMPLE 12

### Sequence Listing for Axl-Specific Antibodies

234
YYCGTADKRDYHSQVFYKPLTGVVDNILTKIMTKHKQIIIHRTGTLIIAL
YFWHCFFSSVFFFVDKITMRIGIASELIKSHKRVPRCLGIIRCGVRKRLF
LLGSTKIIKLAAKKRLLLQARFVVPLGVLRGLLSAYCCYFALCRP
235
YCGTYQTGTQSMPFSVMLVEIVVDQGTLVTVSSAAPRRSSNRYP
236
YYCGTRDPPSTVTVGSEVSVDLMVDQGTLVTVSSSANKNSSNRYPDLALY
FWHCFFSWIFFFVDNINIRQPMTHMHQPTQITGTVANIVHLSSDDSWSFP
LCVDQNKLISTHRRILIKLNTLGT
237
YYCGTYQTGTQSMPFSVMLVEIVVDQGTLVTVSSAAPRRKRRGTG
244
YYCGTWDGRCQYQPRVHFRVGGLVDQ
250
YYCGTALLDVGPKSAHVAVSTLQVDIIIQTHKRKINKKNIQKKGTRNLTS
GRGKYAPNWICDSVDSTNIRQPMTHMHQPTKITGTVANIVQLSSDDSWSF
RLCVDQNKLKSTHRRILKKLNTLGTL
258
YYCGTSRDQGVLFKFCVSPSIFYVDRHILQRRPSRIKPTQRTSGTGALQQ
NMQLKYQTSGHVHVDQ

Although the invention has been explained in relation to its preferred embodiments and working examples, it is to be understood that many other possible modifications and variations can be made without departing from the spirit and scope of the invention as herein described.

What is claimed is:

1. A phage display library, comprising
a plurality of recombinant phages;
each of the plurality of recombinant phages comprising an M13-derived expression vector;
the M13-derived expression vector comprising a polynucleotide sequence encoding a randomized peptide, wherein the randomized peptide is a variable region of a human IgG1 heavy chain; and
the randomized peptide is expressed in absence of an immunoglobulin light chain protein on an outer surface of a recombinant phage of the phage display antibody library.

2. The phage display library as claimed in claim 1, wherein the randomized peptide comprises a human complementarity determining region 1 (CDR1) or a human complementarity determining region 2 (CDR2).

3. The phage display library as claimed in claim 1, wherein the randomized peptide comprises a human complementarity determining region 3 (CDR3).

4. The phage display library as claimed in claim 2, wherein the randomized peptide consists of a plurality of glycine and a plurality of serine.

5. The phage display library as claimed in claim 1, wherein the randomized polypeptide is derived from NNK oligonucleotides and is flanked on 5′ end thereof with a Kpn I restriction site and on 3′ end thereof with a Sal I restriction site.

6. The phage display library as claimed in claim 1, wherein the randomized polypeptide is derived from NNK oligonucleotides and is flanked on 5′ end thereof with a Bsa I restriction site and on 3′ end thereof with a Sal I restriction site.

7. The phage display library as claimed in claim 1, wherein the randomized polypeptide is derived from NNK oligonucleotides and is flanked on 5′ end thereof with a Sac I restriction site and on 3′ end thereof with a Xba I restriction site.

8. The phage display library as claimed in claim 1, wherein a protein product expressed by a recombinant phage of the phage display antibody library fuses with a human immunoglobulin constant region to form a full human IgG1 heavy chain molecule.

9. The phage display library as claimed in claim 8, wherein the full human IgG1 heavy chain molecule is further combined with a full human light chain molecule to form a complete human antibody.

10. The phage display library as claimed in claim 9, wherein the complete human antibody binds to a cell surface receptor selected from the group consisting of MerRT, cMet, EGFR, ErbB3, PDGFR and Axl.

11. The phage display library as claimed in claim 10, wherein the cell surface receptor is Axl.

12. A phage display library, comprising
a plurality of recombinant phages;
each of the plurality of recombinant phages comprising an M13-derived expression vector;
the M13-derived expression vector comprising a polynucleotide sequence encoding a randomized peptide, wherein the randomized peptide is a variable region of a human IgG1 heavy chain;
the randomized peptide is expressed in absence of an immunoglobulin light chain protein on an outer surface of a recombinant phage of the phage display antibody library; and
a protein product expressed by a recombinant phage of the phage display antibody library fuses with a human immunoglobulin constant region to form a full human IgG1 heavy chain molecule.

13. The phage display library as claimed in claim 12, wherein the randomized peptide comprises a human complementarity determining region 1 (CDR1) or a human complementarity determining region 2 (CDR2).

14. The phage display library as claimed in claim 12, wherein the randomized peptide comprises a human complementarity determining region 3 (CDR3).

15. The phage display library as claimed in claim 13, wherein the randomized peptide consists of a plurality of glycine and a plurality of serine.

16. The phage display library as claimed in claim 12, wherein the randomized polypeptide is derived from NNK oligonucleotides and is flanked on 5′ end thereof with a Kpn I restriction site and on 3′ end thereof with a Sal I restriction site.

17. The phage display library as claimed in claim 12, wherein the randomized polypeptide is derived from NNK oligonucleotides and is flanked on 5′ end thereof with a BsaI restriction site and on 3′ end thereof with a Sal I restriction site.

18. The phage display library as claimed in claim 12, wherein the randomized polypeptide is derived from NNK oligonucleotides and is flanked on 5′ end thereof with a Sac I restriction site and on 3′ end thereof with a Xba I restriction site.

19. The phage display library as claimed in claim 12, wherein the full human IgG1 heavy chain molecule is further combined with a full human light chain molecule to form a complete human antibody.

20. The phage display library as claimed in claim 12, wherein the complete human antibody binds to a cell surface receptor selected from the group consisting of MerRT, cMet, EGFR, ErbB3, PDGFR and Axl.
